(19) United States
(12) Plant Patent Application Publication
(10) Pub. No.: US 2005/0091720 P1
(43) Pub. Date: Apr. 28, 2005

(54) Seedless grape

(50) VITIS VINIFERA; GRAPE

(76) Inventors: Robert Garry Trezise, Paringa (AU); Peter Michael Burne, Renmark (AU)

Correspondence Address: KLAUBER & JACKSON, 411 HACKENSACK AVENUE, HACKENSACK, NJ, 07601, US

(21) Appl. No.: 10/949,725

(22) Filed: Sep. 24, 2004

(30) Foreign Application Priority Data
Sep. 25, 2003 (AU) 2003905224

(51) Int. Cl. A01H 5/00

(52) U.S. Cl. PLT/205

(57) ABSTRACT

The present invention relates to a seedless grape variety, which is a bud sport of a Chardonnay grape. A seedless variety has considerable advantages in wine-making in that the wine being produced can be left on the skins for longer than in the case of conventional Chardonnay clones, without developing the bitter taste associated with seeds.

The Latin name of the genus and species of the plant claimed herein is Vitis vinifera, cultivar Chardonnay. The plant is a seedless grape variety, which is a bud sport of a Chardonnay grape.

### BACKGROUND OF THE INVENTION

A seedless variety has considerable advantages in wine-making in that the wine being produced can be left on the skins for longer than in the case of conventional Chardonnay clones, without developing the bitter taste associated with seeds.

The improved grape of the present invention was first noticed, in January of 1999, by one of the inventors, who observed a vine having unusually small bunches with small berries in a Chardonnay planting on the property/vineyard of R & M Trezise, Murtho Road, Paringa, South Australia, Australia (CT 5136/331 Hundred Murtho Section 66).

A year later, a viticultural consultant suggested that the vine should be investigated, as it might be of value in winemaking. A test sample of two litres of wine was produced from the grapes. The wine showed potential, despite receiving insufficient attention during the winemaking process.

In the next year's vintage (2001), further tests were conducted. The grapes were found to contain 12 g/l total acid at 13.9 Be, which was a remarkable result. The grapes were made into wine in the laboratory, and this time the wine appeared very promising. The wine analysis was 8.0 g/l total acid, pH was 2.85 (remarkably good), and the alcohol content was 13.9%.

The mother vine was DNA typed by the Australian Wine Research Institute, using six microsatellite loci, and found to match the genotype of material designated as Chardonnay, as recorded in the databases.

During the dormancy period of 2001, the mother vine was tested by Waite Diagnostics and no vitiviruses or phytoplasmas were detected.

In spring of 2001, sufficient vines were propagated from the mother vine to plant two 100-metre-long rows on Paulsen rootstock, and one row on their own roots on the property/vineyard of R & M Trezise, Murtho Road, Paringa, South Australia, Australia (CT 5136/331 Hundred Murtho Section 66). These new vines were regularly examined, through to harvest 2004, for any variation from the mother vine. Three vines each produced an individual cane (shoot) that reverted back to the original Chardonnay. These canes had normal sized berries and bunches and had normal leaves. This reversion rate is within normally accepted tolerances. All the other canes produced on the three vines remained seedless, as did all the canes on the other 190 vines.

Meanwhile, in late 2002, further trials were carried out with a planting of the seedless clone at the Riverland Vine Improvement Committee's Monash property in South Australia. It is covered by a non propagation agreement with the Riverland Vine Improvement Committee.

During vintage 2003, wine was produced from the mother vine and its young progeny. The yield from the mother vine was 31 kg (equivalent to 38.5 t/ha), and winemakers examining the wine produced from the seedless grapes were favourably impressed.

During the 2004 vintage, wine was again produced from the mother and daughter seedless vines, with similar results.

### BRIEF SUMMARY OF THE INVENTION

The present invention relates to a seedless Chardonnay grape, and in particular to a seedless Chardonnay grape having a readily apparent morphological marker. That morphological marker is a characteristic and very distinctive leaf shape, whereby the petiolar sinus forms an open “V”, which serves to “fingerprint” the variety. The seedless Chardonnay grape specifically described herein is a bud sport of FPMS6 Chardonnay, and can be readily distinguished from all other Chardonnay clones by its unique leaf marker, even before examining its equally distinctive bunches and berries. It can therefore be easily identified. As far as the applicants are aware, that is not possible for any other Chardonnay clones, and currently not even DNA analysis can distinguish between clones of Vitis vinifera varieties.

This seedless Chardonnay sport was originally produced from FPMS6 Chardonnay grafted to 140 Ruggeri rootstock. FPMS6 is sourced from Foundation Plant Material Services, University of California, Davis, U.S.A. It is readily available from nurseries. The seedless Chardonnay sport has the following characteristics:
It has been DNA tested to be Chardonnay by the Australian Wine Research Institute.
It produces wine with characteristic Chardonnay flavours and bouquet.
Bud bursts are in spring, at nearly the same time as known Chardonnay FPMS6 and other clones. For example, in 2004, bud bursts for the seedless Chardonnay were just 6 days later than for conventional Chardonnay.
It matures at the same time as Chardonnay, and has the same growing habit as Chardonnay.
It produces white grapes that have the distinctive flavours that characterise most other Chardonnay clones.
It produces bunches that have all the hallmarks of Chardonnay, except that they are approximately one-third the weight (ie about 55 g, compared to about 145 g).
It produces berries that are much smaller than FPMS6 Chardonnay berries (ie each berry weighs around 0.44 g, as compared to a weight of around 1.3 g for FPMS6 Chardonnay).
It produces berries that contain, in most cases, aborted seeds although, in a very small number of cases, there will be the occasional normal-sized seed. To the untrained person, they appear virtually seedless. The term “seedless”, as used throughout the specification and claim, has this meaning, and does not necessarily indicate that the berries completely lack seeds.
The leaves, although otherwise similar to other Chardonnay varieties, have the petiolar sinus forming a distinctive open “V” with slight parallel veining adjacent to the sinus. This enables the clone to be readily distinguished from all other Chardonnay clones.

These distinctive characteristics are clearly shown in the photographs of FIGS. 1, 2 and 3. From the presence of this morphological marker, being the characteristic and very distinctive leaf shape, it can be surmised that the gene for the seedless characteristic is closely linked to that for the leaf shape marker.

The seedless gene mutation is unusual, in that the berries of the seedless Chardonnay still set well.

The berries of the present variety have a higher acid content than that of conventional Chardonnay varieties at the same stage of maturity. This is a particular advantage for grapes grown in high temperature regions, where low acid content seems to be a problem.

The present variety tends to produce much smaller bunches and much smaller berries than other Chardonnay clones. The smaller size of the berries means that there will be a higher ratio of skin to fruit volume. However, this is not a problem, because the lack of seeds means that there are still similar proportions of flesh. In fact, the higher ratio of skin to fruit volume can be advantageous, in that some significant flavour components are mainly present in the vicinity of the skin.

### BRIEF DESCRIPTION OF THE DRAWINGS

FIG. 1 is a side-by-side comparison of bunches of Chardonnay FPMS6 grapes, and the leaves of that variety, with bunches of the present seedless grape, and the leaves of this new clone. (I10V1 is the Australian code for FPMS6.)

FIG. 2 is a comparison of a bunch and leaf of the new variety (foreground) with bunches and leaves of a FPMS6 Chardonnay vine (background).

FIG. 3 is a photograph of the new vine.

### DETAILED BOTANICAL DESCRIPTION

Various tests have been carried out to characterise the seedless Chardonnay of the present invention.

Firstly, the morphological characteristics have been observed.

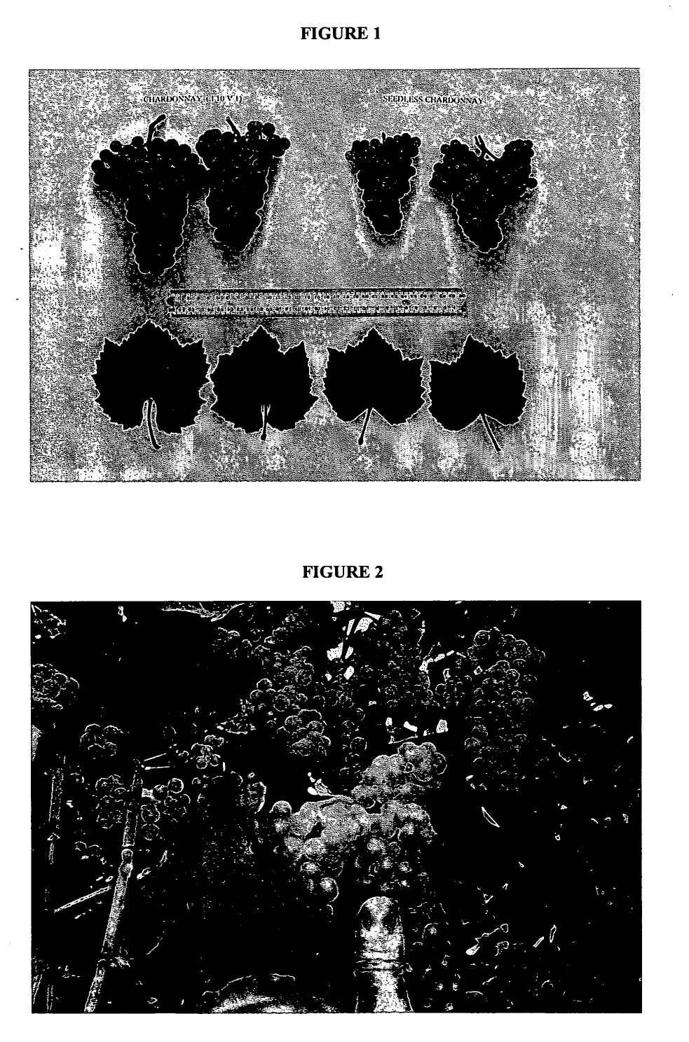

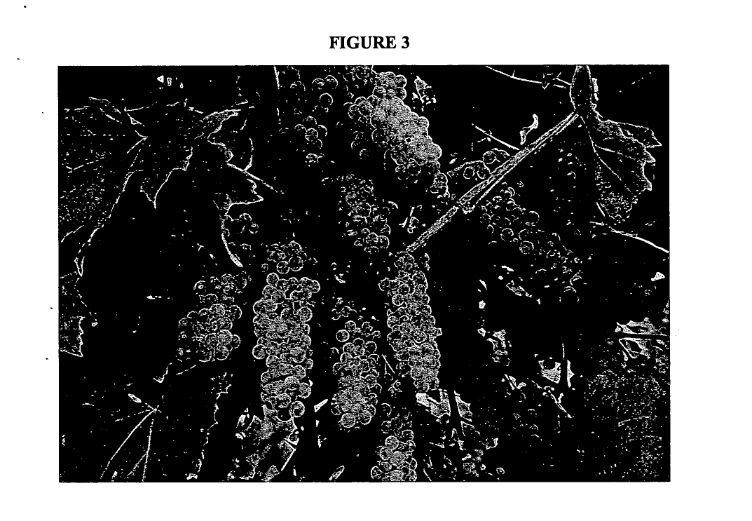

Most significantly, there is a morphological marker, being the distinctive and characteristic leaf shape. This is shown in FIG. 1, where the leaf shape of the seedless Chardonnay is compared to that of the parent variety (Chardonnay FPMS6). The petiolar sinus of the leaf of the seedless Chardonnay is a distinctive open “V” with slight parallel veining adjacent to the sinus.

The seedless variety has the same growing habit as Chardonnay, and produces bunches of white grapes which have all the hallmarks of Chardonnay, except that the bunches are approximately one-third the weight (ie around 55 g, compared to around 145 g). The berries are much smaller than FPMS6 Chardonnay berries (ie about 0.44 g, as compared to about 1.3 g). The berries appear to be virtually seedless.

A sample of the seedless Chardonnay was DNA typed by the Australian Wine Research Institute, using six microsatellite loci. Its DNA profile was compared with DNA profiles of other grapevine material contained in a database of the Australian Wine Research Institute. The source grapevine material used to produce the database was drawn from collections held by the Commonwealth Scientific and Industrial Research Organisation (CSIRO) at Merbein, Victoria, and by the South Australian Research and Development Institute at Nurioopta. This material, in turn, was originally received from overseas countries, together with a description of the material as representing a certain named variety of grapevine. Subsequently, most of that material (in particular, the material held by CSIRO at Merbein) was subject to independent expert ampelographic verification. The genotype of the seedless variety of the present invention was found to match the genotype of the material designated as Chardonnay in the database.

Samples of the seedless Chardonnay, supplied as dormant canes, were PCR tested by Waite Diagnostics for the presence of the following viruses and phytoplasmas: grapevine leafroll-associated viruses 1 to 5, Rupestris stem pitting associated viruses 1 and 2, Grapevine Vitiviruses A, B and D, grapevine fleck viruses A and B, Grapevine Fanleaf virus and phytoplasmas (including Australian Grapevine Yellows). None were detected.

The berries of the seedless Chardonnay were found to have a lower pH than FPMS6 Chardonnay at the same stage of maturity.

Because the berries are virtually seedless, less tannins are present. The berries also have a relatively low phenolic content.

During the 2002 vintage, a winemaking trial was conducted, in which the seedless Chardonnay and a seeded Chardonnay variety were compared. The trial yielded the following data:

Fruit Weight (Total Delivered)

Seedless: 33.65 kg

Seeded: 119.35 kg

Bunch Weight (Based on a Sample of 62-77 Bunches)

Seedless: 69.48 g

Seeded: 113.71 g

Berry Weight (Based on a Sample of a 195-198 Berry Bunch)

Seedless: 0.483 g

Seeded: 1.149 g

Extraction Rate (NB: Pressing was conducted using a water bag press applying a pressure of 225 kPa—greater extraction rates would probably be achievable under commercial conditions using greater quantities of fruit)

Seedless:
594 L/tonne (unclarified following pressing)
535 L/tonne (clarified following racking)

Seeded:
595 L/tonne (unclarified following pressing)
536 L/tonne (clarified following racking)
Juice Analysis (NB: Fruit was stored at 0° C. for a period of 72 hours prior to crushing. A substantial amount of tartaric acid may have been lost during this period due to potassium bitartrate precipitation)

$$\begin{matrix}\text{Seedless:} \\ \begin{matrix}\text{Total Soluble Solids} = {23.0}^{0} Brix , {12.8}^{0} Buame \\ pH = 3.16 \\ \text{Titratable Acidity} = 6.6 \text{g/L} as tartaric acid\end{matrix} \\ \text{Seeded:} \\ \begin{matrix}\text{Total Soluble Solids} = {23.4}^{0} Brix , {13.0}^{0} Buame \\ pH = 3.38 \\ \text{Titratable Acidity} = 4.58 \text{g/L} as tartaric acid\end{matrix}\end{matrix}$$

1. A new and distinct variety of seedless grape plant, substantially as illustrated and described herein.
